(19) United States
(12) Patent Application Publication
(10) Pub. No.: US 2024/0252306 A1
(43) Pub. Date: Aug. 1, 2024

(54) TIGON MEDICAL BUTTON SYSTEM

(71) Applicant: TIGON MEDICAL, Millersville, MD (US)

(72) Inventors: Dan Pyle, Millersville, MD (US); Jeremy Clark, Millersville, MD (US); Gabe Horneff, Millersville, MD (US); Umasuthan SRIKUMARAN, Ellicott City, MD (US)

(21) Appl. No.: 18/629,830

(22) Filed: Apr. 8, 2024

Related U.S. Application Data
(63) Continuation-in-part of application No. 17/746,426, filed on May 17, 2022.
(60) Provisional application No. 63/181,906, filed on Apr. 29, 2021.

(51) Int. Cl. A61F 2/08

(52) U.S. Cl. CPC A61F 2/0805; A61F 2002/0817; A61F 2002/0852; A61F 2002/0882; A61F 2220/0033

(57) ABSTRACT

Disclosed are improved methods, apparatus and/or systems for securing and/or anchoring tissue structures to bones, including unicortical soft tissue fixation.

### CROSS-REFERENCE TO RELATED APPLICATIONS

This application is a continuation-in-part of U.S. patent application Ser. No. 17/746,426 entitled “TIGON MEDICAL BUTTON SYSTEM,” filed May 17, 2022, which claims the benefit of U.S. Provisional Patent Application Ser. No. 63/181,906 entitled “TIGON MEDICAL BUTTON SYSTEM,” filed Apr. 29, 2021, the disclosures of which are incorporated by reference herein in their entireties.

### TECHNICAL FIELD

The invention relates to improved orthopedic tools and methods for use during orthopedic surgical procedures, including joint repair and/or replacement procedures. More specifically, disclosed are improved methods, apparatus and/or systems for securing and/or anchoring tissue structures to bones, including unicortical soft tissue fixation.

### BACKGROUND

There are a wide variety of suture anchor designs, related devices and/or surgical techniques for securing sutures and/or tissue grafts, which anchors and/or sutures can be passed through soft tissue and/or bones. A graft fixation member, e.g., a fixation button, can be passed through a bone tunnel and used to secure a tissue graft and/or suture, such as the device described in U.S. Pat. No. 5,306,301, hereby incorporated by reference in its entirety. While there are a wide variety of fixation buttons commercially available on the market, there is need for further improvement in surgical implants of this type, and the present subject matter is such improvement.

### SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with various aspects of the present subject matter, systems, devices and/or methods are disclosed which may be useful in orthopedic procedures which require attachment and/or anchoring of tissues to bones or other anatomical features. Various embodiments disclose a flexible tether-based tension fixation system, wherein a first anchor (which may comprise a relatively rigid, elongated portion or “button”) which can be introduced into and through a bone tunnel, with the anchor rotated to present a profile that is larger than the tunnel diameter, and the flexible tether is connected to a tissue structure and/or tensioned to draw the anchor against the bone. Properly employed, the disclosed system can secure a wide variety of tissues to repair and/or facilitate normal function of the patient's anatomy and also reduce recovery time, including attachment to virtually any natural and/or artificial tissue. In addition to the anchoring components described herein, surgical tools are disclosed that facilitate implantation and deployment of the anchoring devices and related components.

In various applications, the disclosed implant components and/or associated devices can comprise various medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, plastics, apatites and/or various 3D printed materials. In some embodiments, an employment of mixed materials in a given implant construction may enhance the strength and/or durability of a desired implant design, as well as allow for improved surgical outcomes and/or greatly reduced complication rates.

If desired, implant components could be constructed from a variety of modular components, including modular components comprising different materials. If desired, device components could be provided in a kit form for selection and/or assembly in a surgical theatre and/or in situ during a surgical procedure, which might include a plurality of buttons of varying shapes and/or sizes within a single surgical kit. If desired, various components may be removable and replaceable.

Various surgical methods for implanting or placement of the various devices and/or components described herein are also described, including the insertion and placement of implants into and/or through a bone tunnel that is formed into a bone, including bones that may be adjacent to and/or within one or more joint surfaces.

In accordance with another aspect of the present subject matter, various methods for manufacturing devices and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings

### BRIEF DESCRIPTION OF THE DRAWINGS

The foregoing and other features and advantages of the present subject matter will become apparent to those skilled in the art to which the present subject matter relates upon reading the following description with reference to the accompanying drawings:

FIGS. 1A through 1G depict various views of one exemplary embodiment of an anchor button constructed in accordance with various teachings of the present invention;

FIGS. 2A through 2F depict various alternative views of an anchor button constructed in accordance with various teachings of the present invention;

FIGS. 3A and 3B depict disassembled and assembled views, respectively, of an insertion tool for inserting and deploying the button of FIG. 1A during a surgical procedure;

FIG. 4 depicts insertion of an inserter into a handle;

FIG. 5A depicts placement of a button on the inserter of FIG. 4;

FIG. 5B depicts a button secured to the inserter of FIG. 5A;

FIG. 5C depicts a button and attached inserter, including externally visible alignment markings on the inserter shaft;

FIG. 5D depicts a plurality of sutures inserted through the fixation openings of the button of FIG. 5B;

FIGS. 6A through 6C depict various exemplary steps of implanting an anchor button in an exemplary implantation procedure;

FIGS. 7A through 7H depict various additional steps of implanting an anchor button in an exemplary implantation procedure;

FIGS. 8A through 8D depict cross-sectional views of a treated bone with a button being manipulated therein;

FIG. 9 depicts a perspective view of another alternative embodiment of a button which includes a distal through hole;

FIG. 10 depicts one exemplary embodiment of a suture loop or lasso;

FIG. 11A through 11C depict various views of a pair of suture loops extending through openings in a button;

FIGS. 12A through 12D depict various views of one exemplary embodiment of a button caddy or containment device;

FIGS. 13A and 13B depict top plan views of an exemplary button positioned within a slot of a caddy;

FIG. 13C depicts a side cross-sectional view of the implant and caddy slot of FIGS. 13A and 13B;

FIGS. 14A depicts a partial cross-sectional view of an inserter and button properly aligned within a cavity of a caddy;

FIGS. 14B and 14C depict partial cross-sectional views of an inserter and button that are not substantially aligned;

FIG. 15A depicts a top plan view of a button properly aligned within a cavity of a caddy; and

FIGS. 15B and 15C depict top plan and side cross-sectional views of a button that is not properly aligned within a cavity of a caddy.

### DETAILED DESCRIPTION

Various features of the present invention include the recognition of a need for a more effective and versatile system of fixating and/or securing soft tissues to bony structures in a human body. A variety of configurations, sizes and/or shapes of such components and associated tools can be utilized in various diverse anatomical regions. In various medical applications, the disclosed components and related surgical tools and techniques can desirably facilitate the treatment of various tissue injuries, which can be important to achieve the most accurate and best performance and/or fit of implant components and well as facilitate patient recovery.

This specification describes novel systems, devices and methods to anchor tissues and/or sutures to bone. Aspects of the present invention will be described with regard to the treatment of tendon and bicep repair. It should be appreciated, however, that various aspects of the present invention may not limited in their application. The systems and methods may be applicable to the treatment of various tissues and/or diverse bone types. Embodiments will now be described with reference to the drawings, wherein like reference numerals are used to refer to like elements throughout. It should be understood that the figures are not necessarily to scale.

In various embodiments, the disclosed devices, systems and methods can be used as a substitute for various existing screw-based fixation techniques and implants for soft tissue anchoring, including various surgical techniques, tools and related implants described herein. Various embodiments disclose a flexible tether based tension/compression fixation system, wherein a first anchor (which may comprise a relatively rigid, elongated portion or “button”) can be introduced into and through a bone tunnel, with the first anchor rotated to present a profile that is larger than the tunnel diameter, and the flexible tether is connected to a tissue structure and/or tensioned to draw the first anchor into intimate contact with portions of the bone.

Properly employed, the disclosed system can secure a wide variety of tissues to repair and/or facilitate normal function of the patient's anatomy and also reduce recovery time, including virtually any natural and/or artificial tissue, including biological tissues, ligaments, tendons, bone, and cartilage as well as soft tissue grafts and/or engineered tissues such as Tigoderm, etc.

In various embodiments, the various system components may further provide for manipulation, modification and/or even minimally-invasive removal of the button and/or associated and associated fixation components, in the event such activities and/or removal may be required during the surgical implantation procedure and/or in the future.

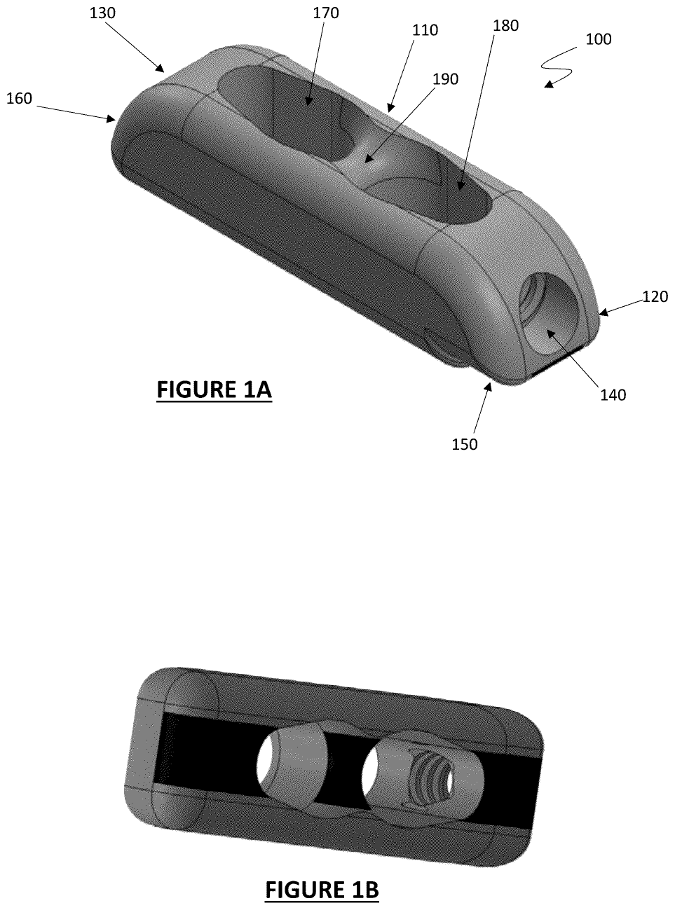

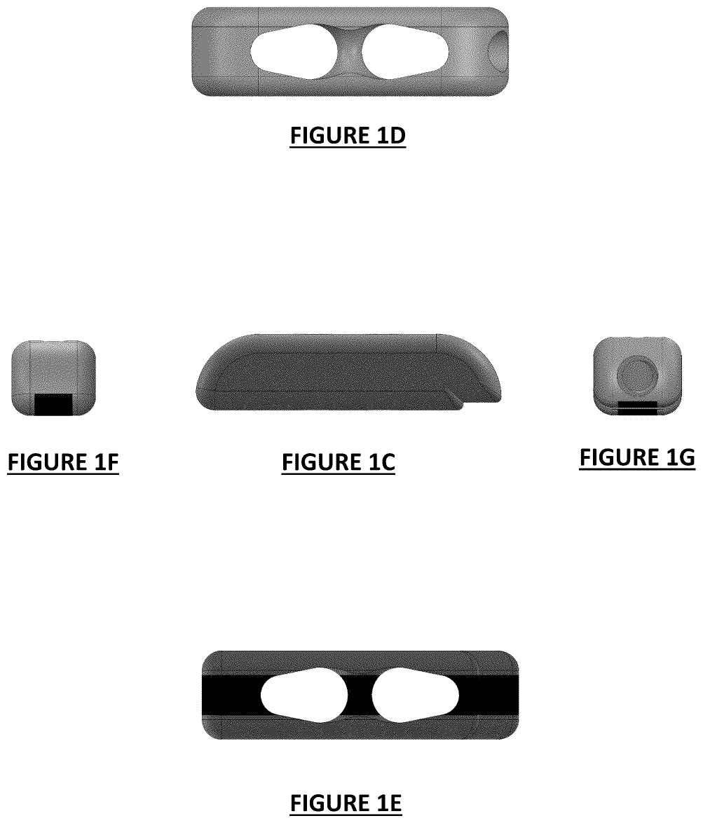

FIGS. 1A through 1G depict various views of one exemplary embodiment of an anchor button 100 constructed in accordance with various teachings of the present invention. The anchor button 100 comprises an elongated body 110 with a proximal end 120 and a distal end 130. The proximal end 120 presents a gradually curved or tapered profile, with the proximal end including a tool opening 140 and lower notched section 150. The distal end 130 similarly presents a gradually tapered profile, with the distal end terminating at a lower end in a generally blunt distal tip 160. A plurality of fixation openings 170 and 180 are formed in the elongated body 110, through which a flexible fixation member (not shown) can extend for placement and tensioning of the construct. A recessed groove member 190 extends between the fixation openings 170 and 180, with this groove member formed into an upper surface of the elongated body 110. The groove member 190 also includes a recessed lower surface, as best seen in FIG. 1B.

In the disclosed embodiment, the downward curving nature of the proximal and distal faces of the button may also be particularly useful in that these structures can reduce and/or eliminate irritation to soft tissue when the button is placed bicortically. Moreover, the construction of the proximal surface can additionally allow the button to “fall away” from a matching curvature surface of the inserter in a consistent and repeatable manner, as discussed below.

While the recessed groove member 190 is shown as a gently curved convex surface, it should be understood that other shapes, including shapes having greater curvatures and/or lesser curvatures (and/or three-dimensional curvatures, if desired), as well as flat surfaces and/or angled surfaces, could be incorporated therein. Similarly, in alternative embodiments the upper and lower portions of the recessed groove member 190 could comprise a concave surface, and/or various combinations of concave, convex, flat and/or angled surfaces, if desired. Desirably, each of the fixation openings will incorporate rounded or tapered edges to reduce and/or eliminate the likelihood of damaging the flexible members which extend therethrough. It should be understood that some or all of the openings in the buttons described herein could be countersunk or otherwise rounded or tapered so as to allow easier threading passage of the flexible members and to reduce the potential for severing and/or fatigue fracture of the flexible members under loading conditions.

In various embodiments, a button (such as the various embodiments shown herein) may have any suitable dimension (diameter and thickness), as well as any suitable number of openings (i.e., 1 or 2 or 3 or 4 or more). For example, one exemplary button embodiment might have a height of 3 mm, a width of about 3 mm and a longitudinal length of about 8 mm, which would desirably permit the button implant to be advanced through a 3.2 mm drill hole (i.e., a “bone tunnel”) or other opening using a bone tunnel forming technique such as punching, grinding, awling and/or other manner of bone cutting commonly known in the art. In the disclosed embodiment, the centers of the fixation openings could be about 2 mm from the center of the button, and the centers of the pair of fixation openings could lie substantially along a longitudinal axis passing through the center of the button. The fixation openings of the button can be elongated, as depicted in FIGS. 1D and 1E, or alternatively the fixation openings may have any alternative shape, including shapes where each fixation opening may be desirably equidistant from the center of the elongated body 110. One preferred embodiment can be a fixation opening which is substantially pear or egg-shaped (see FIG. 1E), while other embodiments may be round or oval in plain view. Other exemplary embodiments could include buttons having a longitudinal length that can be more than twice the width of the button and/or having a longitudinal length that may be more than twice the height of the button.

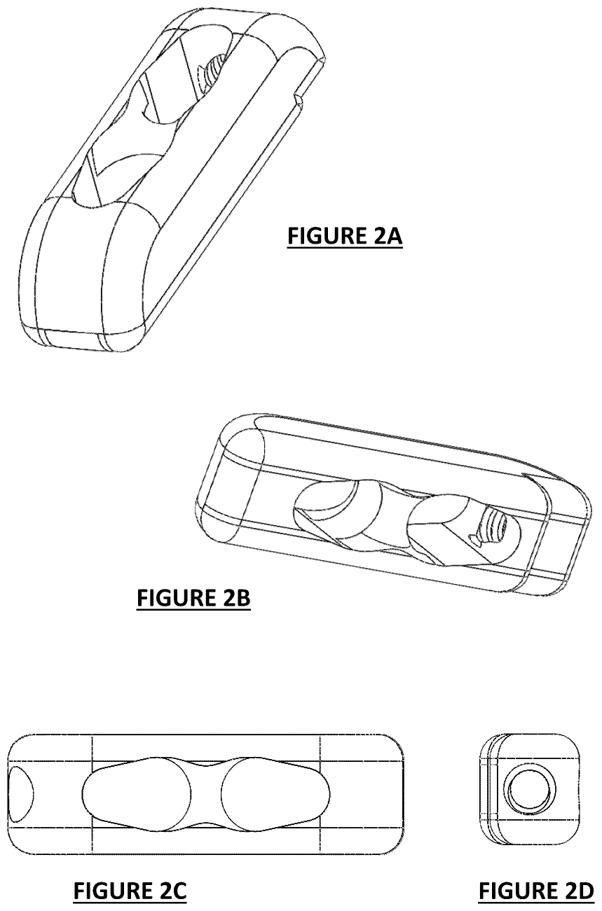

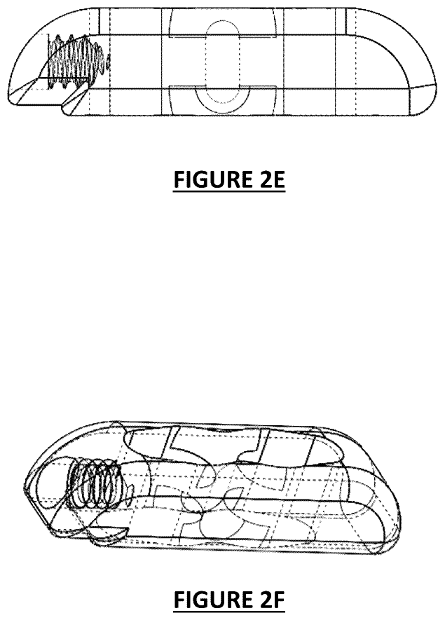

FIGS. 2A through 2F depicts various alternative views of an anchor button constructed in accordance with various teachings of the present invention.

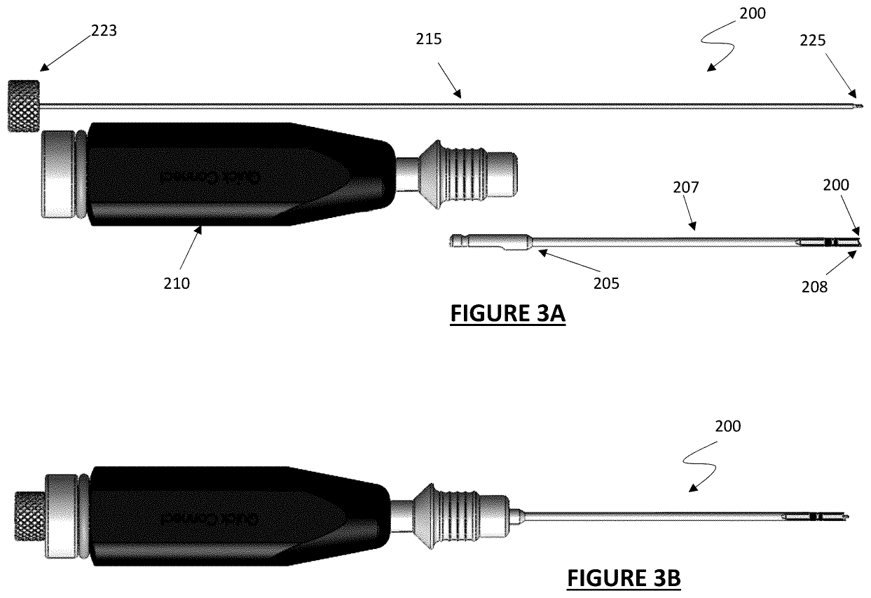

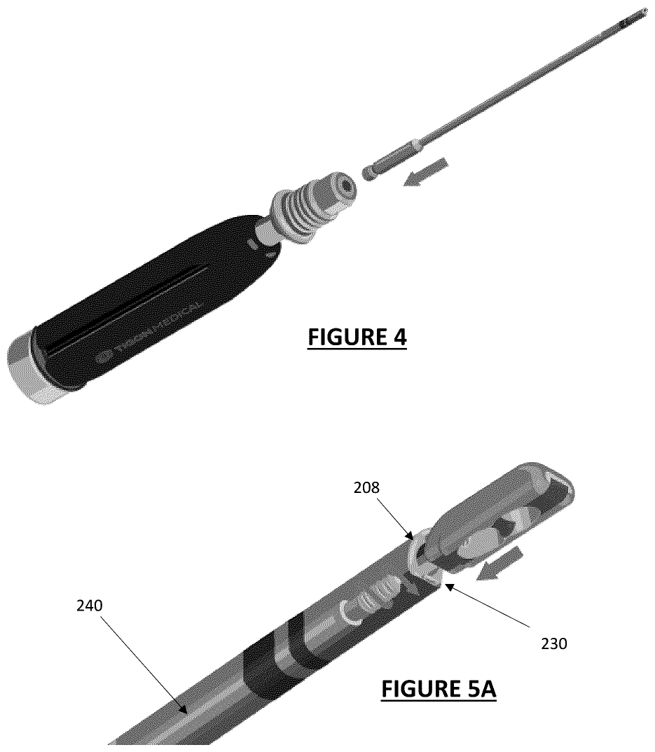

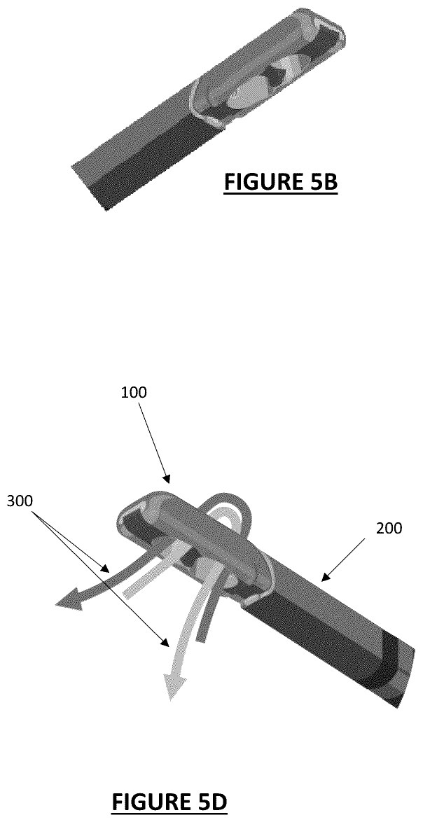

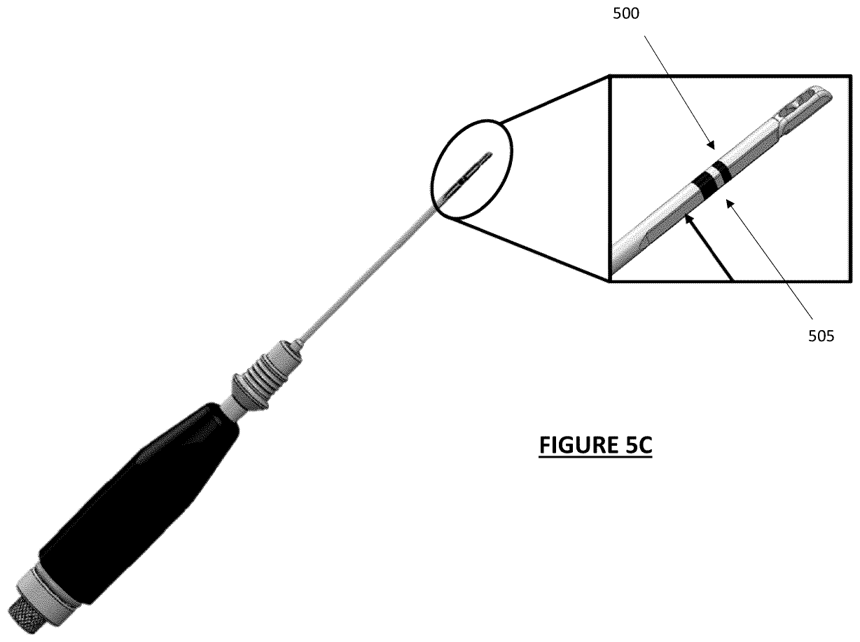

FIGS. 3A and 3B depict disassembled and assembled views of an insertion tool 200 for inserting and deploying the button of FIG. 1A during a surgical procedure. The insertion tool 200 desirably includes an inserter 205 which can be attached to a handle 210 (See FIG. 4). As best seen in FIGS. 3A and 5A, the inserter 205 includes an elongated cannulated shell 207, with the shell including a curved distal tip 208 having a distal alignment feature or tooth 230 positioned proximate to the end of the inserter 205. An internal rotatable shaft 215 is positioned within the cannulation, the shaft 215 including a proximal knob 223 and a distal threaded tip 225, wherein some portion of the shaft 215 and threaded tip 225 are sized and configured to extend outward from the shell 207. FIG. 3B depicts the inserter, handle and shaft in a fully assembled configuration.

In preparation for use, a button can be positioned against the inserter 205, where the curved distal tip 208 of the shell 207 desirably engages with a proximal end of the button, with the lower notched section of the button engaging with the tooth 230 (which desirably concurrently aligns the flattened sides of the button with corresponding flattened sides 240 of the inserter, also allowing suture to slide past the inserter within the small drill hole diameter). In various embodiments, a counter bore of the tool opening can be slightly larger than inner shaft diameter, with an axis of the counter bore and the inner inserter being coaxial. The threaded tip 225 and internal rotatable shaft are then extended into the tool opening, with the shaft 215 rotated to cause the threaded tip 225 to engage with internal threads within the tool opening 140, thereby securing the button 100 onto the inserter 205, as shown in FIG. 5B, and aligning the longitudinal axis of the bullet with the insertion tool and holding these components together in a relatively rigid fashion. Desirably, the inner surface of the curved distal tip 208 can have a shape that matches and/or compliments the outer shape of the proximal end of the button, which desirably engages with and secures the button so as to allow the button to be manipulated and/or rotated while advancing and/or withdrawing the insertion tool into and/or out of the targeted anatomy. FIG. 5C depicts a fully secured button and inserter, including externally visible alignment markings 500 and 505 on the inserter shaft.

As best seen in FIG. 5D, one or more sutures 300 can be inserted into and/or through the fixation openings of the button 100, and the insertion tool 200 is now ready to be utilized to install the button and associated suture into a human patient. In various embodiments, the button could have one or more high strength flexible members attached thereto, including suture tape (such as FiberTape® by Arthrex, Inc. of Naples, FL) or pull-through suture strand (such as #2 FiberWire® sold by Arthrex, Inc. of Naples, FL). If desired, a suture or suture tape could be preattached to the button when loaded onto the insertion tool, or alternatively can be added after the button has been attached to the insertion tool.

In some embodiments, the threaded tip may be of sufficient length, with a threaded portion of the button extending a sufficient distance into the eyelet(s), to allow a portion of the threaded tip to contact and/or compress some portion of the suture(s) within one or more of the eyelets of the button, which could desirably reversibly “lock” the suture within the button for a myriad of reasons. In such an arrangement, the threaded tip could be partial unthreaded to allow subsequent suture movement through the eyelets while still maintaining the button and insertion tool in a relatively rigid relationship. Once the threaded tip was fully unthreaded, however, the button could be manipulated and deployed as otherwise described herein.

One surgical repair well suited for use with the disclosed devices is biceps tenodesis, which desirably treats biceps tendon tears caused by injury or overuse. This procedure can also be utilized to treat SLAP tears—i.e., tears in a labrum, which is the cartilage that lines an inner part of the shoulder joint. Biceps tenodesis is accomplished by detaching the biceps tendon from the labrum (or where damage has already detached and/or injured the tendon) and moving the tendon to an upper arm bone called the humerus.

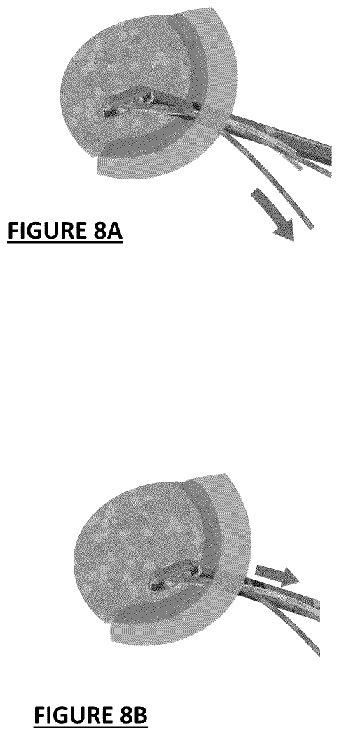

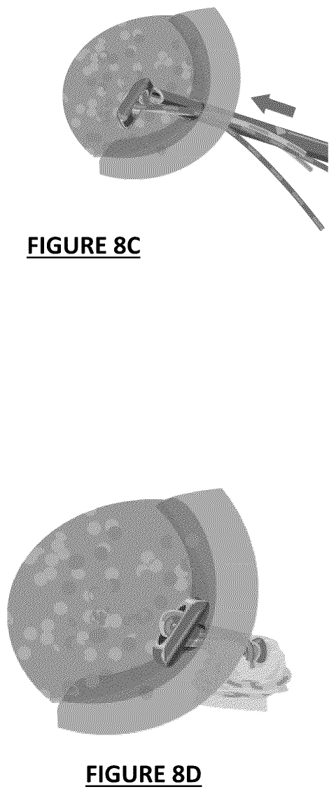

Biceps tenodesis is a common procedure performed for tendinopathy of the long head of the biceps brachii (LHB). Indications include partial-thickness LHB tear, tendon subluxation with or without subscapularis tear, and failed conservative management of bicipital tenosynovitis. In one exemplary surgical procedure utilizing one or more of the disclosed embodiments, a physician may first release and capture the long head of the biceps. Using an appropriate drill size for the selected button, the physician may drill into the subpectoral bicipital groove until the drill completely drills through the first cortical layer and before it reaches the far cortex. The drill can then be removed from the drill from the hole and the tendon can be prepared using the physician's preferred stitching technique. An appropriate button may be selected, which may optionally include pre-loaded sutures and/or the button may be preloaded on to the inserter. If the button is not preloaded, a suture tail may be loaded through the distal and then the proximal eyelet of the button, and another suture tail may be loaded through the proximal and then the distal eyelet of the button. The physician can then insert the button and inserter into the drilled hole up to the circumferential laser line on the inserter. The threaded rod may be unthreaded from the button by rotating the knurled knob counterclockwise. The inner inserter can then be removed from the quick connect handle. While keeping the inserter held at its current depth, the physician or an assistance may firmly grasp and pull suture the suture tail that was passed first through the distal eyelet and then the proximal eyelet (see FIG. 8A). While keeping tension on the suture, the physician may slowly back the inserter out of hole to reveal the circumferential laser line (but preferably not remove the inserter completely from the hole) (see FIG. 8B). The physician may then plunge the inserter back into the hole and pull on the suture, wherein the button will desirably rotate (see FIG. 8C). The inserter may then be removed and the suture released (see FIG. 8D). The physician may now shuttle tendon down the bone by pulling and tensioning the suture, and the suture may be tied down and/or otherwise secured and the procedure completed.

In another exemplary surgical procedure utilizing one or more of the disclosed embodiments, a physician may first capture soft tissue deemed necessary for the surgical repair. Using an appropriate drill size for a selected button, the physician can drill into the desired bone location until the drill completely drills through the first cortical layer and optionally before it reaches the far cortex. The drill may then be removed from the hole and the tendon prepared using a preferred stitching technique. The physician or a surgical assistance may assemble the inserter by inserting the quick connect on the outer shaft into an appropriate receiver in the cannulated quick connect handle before inserting the inner inserter through both components. A selected button may then be fixed to the inner inserter and outer shaft by rotating and securing the threaded portion of the inner shaft into the button (using the threads and locating geometry on the button and inserter). The physician can then load one suture tail through the distal and then the proximal eyelet and load the remaining suture tail through the proximal and then the distal eyelet. The button and inserter can then be advanced into the drilled hole, desirably up to the circumferential laser line indicated on the inserter. The threaded rod can then be unthreaded by rotating the knurled knob counterclockwise, and the inner inserter may be removed from the quick connect handle. While keeping the inserter held at its current depth, the physician can firmly grasp and pull the suture tail that was passed first through the distal eyelet and then the proximal eyelet. While keeping tension on the suture, the physician can slowly back the inserter out of hole to a sufficient depth to reveal the appropriate circumferential laser line (desirably not removing the inserter completely from the hole). The physician can then plunge the inserter back into the hole and pull on the suture, which desirably causes the button to rotate in one or more planes of rotation. The inserter can then be removed and the suture released. If appropriate fixation is achieved, the tendon can be shuttled down the bone by pulling and tensioning the suture, and the suture may be secured in a desired fashion.

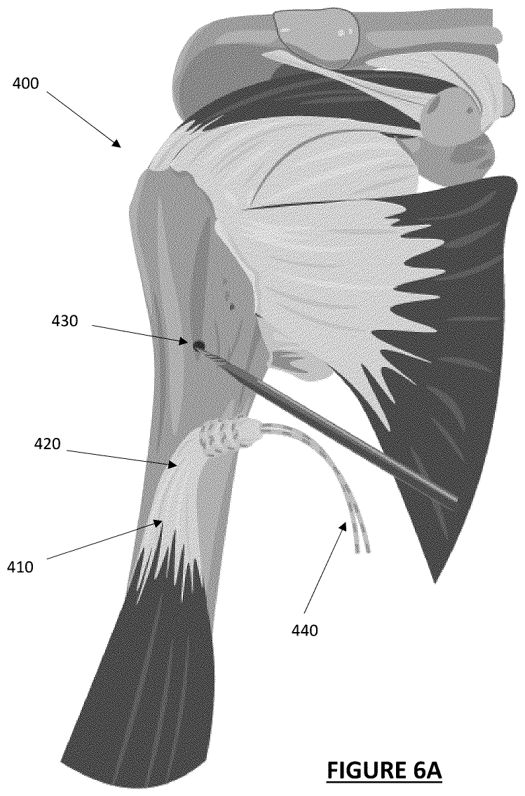

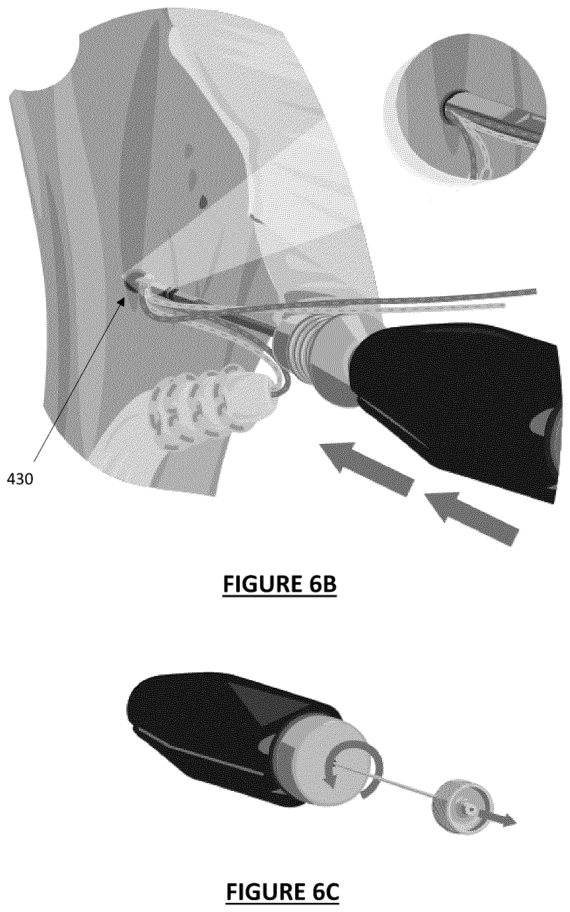

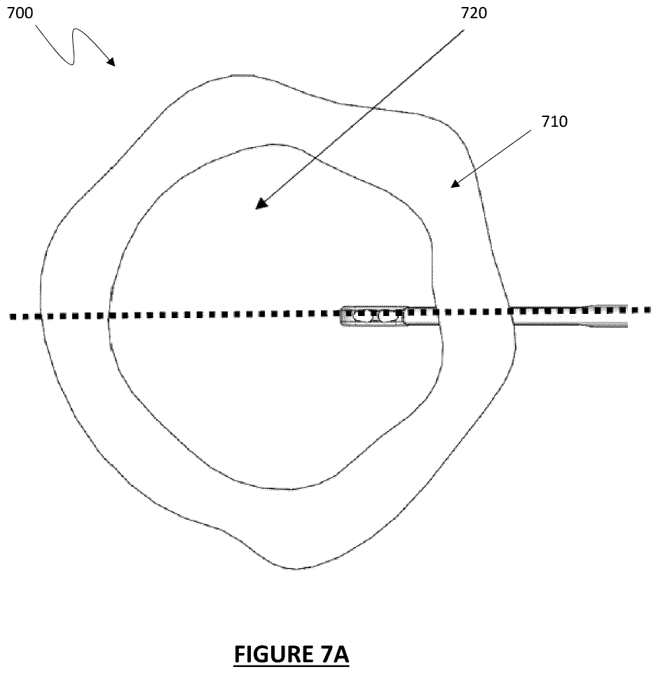

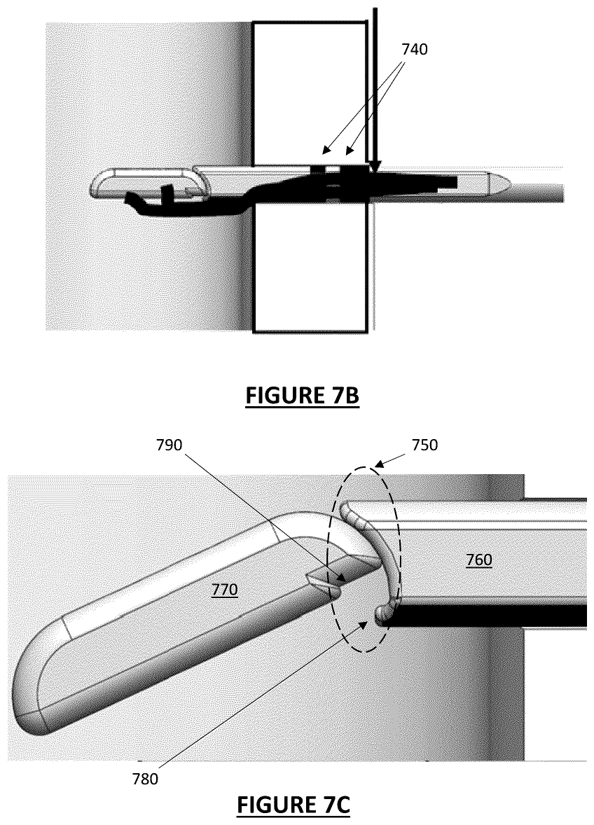

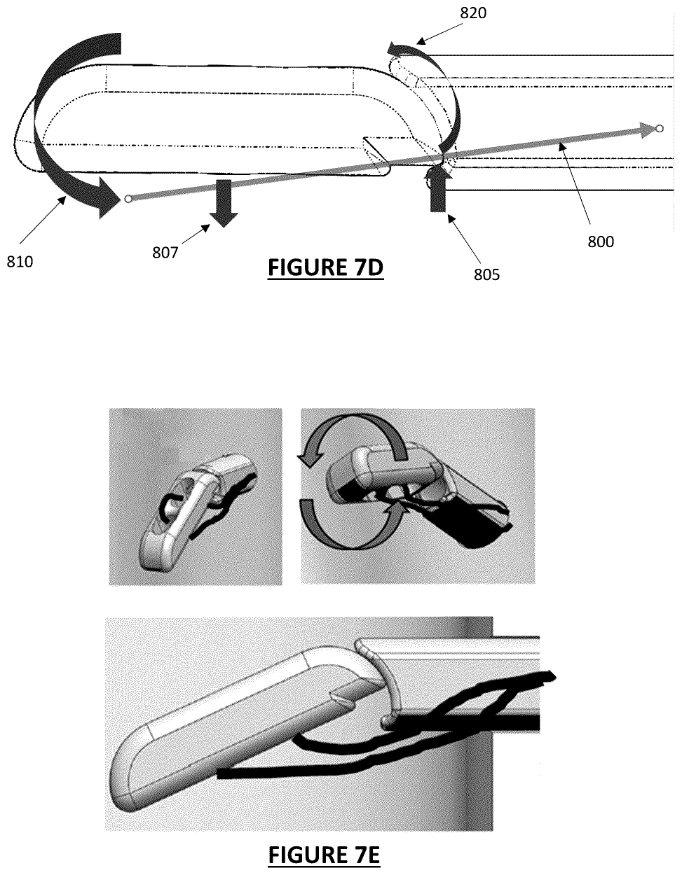

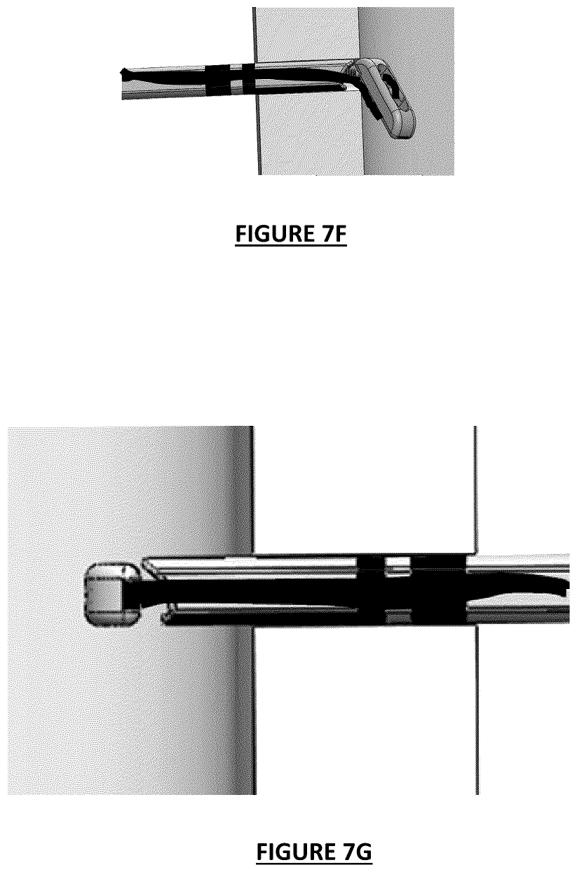

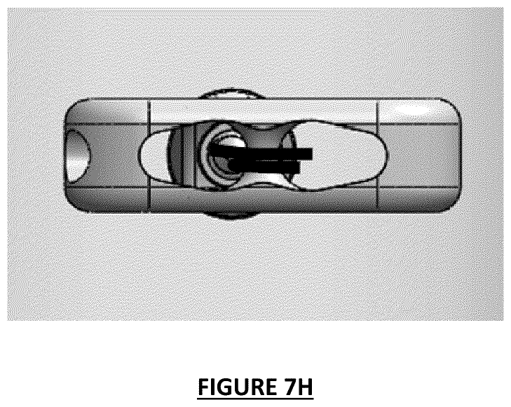

FIGS. 6A through 6C and 7A through 7G depict various exemplary steps of implanting an anchor button in an exemplary bicep repair procedure. FIG. 6A depicts underlying anatomical features of a portion of a patient's shoulder region 400, wherein a biceps muscle 410 and associated biceps tendon 420 have become detached from the shoulder (or where the tendon may have been surgically released from the shoulder for a variety of reasons). In one exemplary procedure, a small hole 430 can be drilled in the humerus (i.e., a single cortex bone tunnel can be drilled), and one or more sutures 440 can be attached to the tendon 420. For example, one or more sutures can be whipstitched or otherwise affixed to a targeted tendon in a surgeon's preferred style, and then passed through the fixation openings of the button. If desired, a sliding limb of the whipstitch can be passed through the proximal eyelet and then the distal eyelet crossing the threshold of the distal eyelet through the flat face and crossing the threshold of the proximal eyelet through the rounded face.

Turning now to FIG. 6B, a button and distal insert tip can be introduced into the small hole 430. As previously noted, the relatively rigid engagement between the button and the insertion tool desirably allows the button and attached insertion tool to be advanced/withdrawn and rotated during advancement and/or retraction until the button reaches a desired position within the targeted anatomy.

FIG. 7A depicts an exemplary bone section 700 comprising a shell 710 of cortical bone surrounding a bone interior 720 of cancellous bone. In various embodiments, one or more laser lines 740 can be provided on the inserter to indicate when the button has transited past the cortical shell and fully entered the cancellous interior (see FIG. 7B), and/or a laser line may be included that indicates when the button is two button lengths (i.e., 16 mm) into the hole. For example, the button can be inserted into the bone tunnel until the first black laser line on the outer inserter is flush with the outer bone surface.

Once the button is in a desired position, the capturing rod can be unthreaded by counter-rotating the internal rotatable shaft to release the button, and the shaft removed from the inserter. Removal of the shaft will desirably allow the button to rotate along the curve of the inserter mount (see FIGS. 7C and 7D). The free suture limb leaving the distal eyelet can then be pulled at approximately a 45 degree angle from the inserter shaft, such that the suture exiting the long face of the button will desirably “slip” off the rounded superior end of the inserter and against the flat (desirably positioned perpendicular to the curved “fish mouth” geometry of the inserter tip), thereby “forcing” or urging the button to twist further relative to the inserter (see FIG. 7E). The outer shaft can then be pressed down to the second laser line and then retreated in the bone tunnel to withdraw the inserter from the bone, which desirably further pulls on the suture and draws the tip of the inserter below the inner cortical wall margin (see FIG. 7F). The inserter can then be reinserted into the cancellous bone region to desirably cause the button to rotate and twist to a final desired orientation (see FIG. 7G). The inserter can then be removed from the bone and the button is now deployed (see FIG. 7H), and the suture can be pulled and/or tensioned to test the hold within the bone and, if the fixation is deemed insufficient, the inserter can be reinserted to desirably reposition the button to attain a desired fixation. Once a desired button fixation is achieved, the suture can be shuttled through the eyelets to pull the tendon down to the bone. The tendon can then be secured with knots and excess suture can be trimmed.

The various design features of the button and the inserter desirably allow the button to be rotated in two separate planes, such as the movements depicted in FIG. 7C (first rotation plane) and FIG. 7E (second rotation plane), which allows the button to be reliably and consistently deployed within the medullary canal of the targeted anatomical bony region. Area 750 in FIG. 7C depicts this “fish mouth” geometry between the outer inserter 760 and the implant 770, where the curvature of these surfaces are substantially complimentary and/or identical in various components. More specifically, these features allow the button to rotate 90 degrees inferiorly and up to 90 degrees from a starting parallel flat condition. Desirably, the outer shaft in some embodiments incorporates complimentary rounded geometry that matches the button geometry, which concentric arrangement includes an outer shaft locating tooth 780 which mates with a corresponding cutout geometry 790 on the implant or button. Because the inner shaft passes through the concentric rounded faces of the outer inserter shaft and the implant or button, engagement of the inner shaft within the button desirably selectively immobilizes and/or rigidifies the button and inserter until such time as the inner shaft is disengaged from the button. In addition, this component arrangement can reduce and/or obviate the potential for the button to “spin” or longitudinally rotate on the thread in an undesirable manner.

Moreover, the locating step feature of the button and inserter allows the inserter to quickly find and align with the button and position itself so that the capturing rod can be quickly and easily threaded proximally on the button. This locating step then functions in tandem with the rounded engaging faces of the inserter and button to rotate the button in a desired direction once the capture rod is detached. The rounded faces can interact to guide the button's rotation around the fulcrum point created by the locating step, and the design and geometry of the button and associated inserter structures facilitates the movement and “flipping” of the button in the same desired and predictable manner. This fulcrum point desirably allows the surgeon to leverage more force to flip the button through the bone that is closest to the medullary cavity. If desired, the caddy and/or insertion tool may incorporate an indicator or manufacturer logo that indicates the inserter is properly aligned with caddy to attach the button thereto.

As best seen in FIG. 7D, rotation of the button begins by pulling on the free suture limb coming from the distal eyelet, schematically indicated as 800. This pulling or tension force pulls the button against the locating feature or “tooth” of the inserter, which acts as a fulcrum point generating a “holding force” 805 which resists button rotation, which resists a normal force 807 induced by this tension which causes the distal end of the button to begin to rotate about the fulcrum, depicted as rotational arrow 810. As the distal tip of the implant rotates away from the longitudinal axis of the inserted (e.g., downward, in this figure, along a first axis of rotation), continued tension on the free suture limb causes the curved surfaces of the implant and inserter to begin sliding relative to each other, depicted as rotational arrow 820. Further tension on the free suture limb eventually displaces the distal tip of the implant towards the entry opening to some limited degree, but desirably draws the center of the implant towards the entry opening, causing the implant to flip in such a way that neither ends of the implant are oriented coaxially to the direction of pull on the suture to the hole it was deployed from. This is because the flat section of the outer inserter desirably funnels the suture attached to the implant perpendicular to the face of the flat on the outer inserter, thereby generating a second axis of rotation of the implant. Ultimately, the design desirably creates a reliable, repeatable process for deploying the implant from the outer inserter.

Various embodiments disclosed herein can allows for dual or multi-axial rotation of the implant relative to the insertion tool, such as the described flipping mechanism which utilizes tension generated by suture, the unique “fishmouth” geometry, the “flat (7)” lengths of the outer inserter that accommodate the suture in an off-axis alignment, and the implant design. The curvature on the outer inserter can generate implantation rotation when force is applied by the suture to the implant, thus the first axis of rotation (e.g., the presence of the locating tooth and corresponding implant cutout causes the distal portion of the implant-and eventually the entire implant body) to rotate with respect to the inverter along the curvature. Concurrently, the flattened side section(s) of the outer inserter desirably funnels the suture attached to the implant perpendicular to the face of the flat on the outer inserter, thereby generating a twisting or rotation (e.g., second axis of rotation) to the implant relative to the inserter. This combined motion allows the implant to “flip” in such a way that neither of the ends of the implant are oriented coaxially to the direction of pull on the suture to the hole it was deployed from, thus strongly anchoring the implant within the bone tunnel.

Desirably, at least in some embodiments the eyelets will have tapers positioned away from the center of the button in order to better transition the forces experienced by the button and to facilitate sliding tapes through the eyelets. The taper of the eyelets desirably allows more material to press against the bone, while the presence of the bone tunnel desirably prevents bone surface contact at the center of the button. The eyelet can also be widest at the suture “bridge” point which facilitates sliding and reduces pinching between the multiple limbs of suture and the button.

In various embodiments, the various dimensions and/or surface features of the bullet (and/or other relevant features of the implant including the length, diameter, number and placement of fixation holes, etc.) could include patient specific features, including bullet surface features designed to match or approximate the surface of the patient's anatomy, which may include use of non-invasive imaging to model and/or create (i.e., virtually and/or by implant manufacture) an inner surface that matches or substantially matches surfaces of the targeted bone region (including the incorporation of three-dimensional surface features, if desired). If desired, various manufacturing methods, including Computer Aided Design (CAD) and 3-D printing techniques can be utilized to design and/or manufacture a desired button using non-invasive imaging data, such as MRI and/or CT scans.

For various medical applications in bony anatomy, for example, the rounded or curved surfaces of the button may be configured to mimic the contour of an underlying bony surface to which the device is attached and/or adjacent to, or the surface may include features that can prepare the underlying bony surface (i.e., roughened surfaces) and/or include surface features that can osseo-integrate with the bone surface, if desired. In various alternative embodiments, the bullet and button components could incorporate one or more bony ingrowth surfaces, which could allow natural healing and permanent fixation of these components to the bone. Similarly, the flexible members described herein could comprise degradable and/or resorbable materials, if desired.

In various embodiments, an asymmetrical profile of the button can substantially support the system versatility and functionality in a variety of ways. For instance, the full roundness of the top face significantly reduces the likelihood of irritation in bicortical use by eliminating sharp edges while the flat face maximizes surface area contact with the bone.

Another particularly useful feature of the various embodiments disclosed herein is the ability of the system to incorporate components constructed from non-ferrous and/or non-magnetic materials (i.e., plastics and/or ceramics). Unlike screw-based fixation, which often requires the use of high strength metals for the screws and/or related components, the components of the present invention could be constructed from virtually any materials, including plastics, ceramics and/or metals, with various plastic components useful in virtually any environment, even where the use of ferrous materials and/or magnetic devices is prohibited (i.e., in high-energy electrical environments and/or near high-strength magnets such as Magnetic Resonance Imaging machines). If visualization of such plastic or ceramic components was desired, such components could include radiopaque elements and/or marker bands, as is well known in the art. In various exemplary embodiments, the bullets and buttons described herein could comprise titanium, stainless steel, polyethylene, PolyEtherEther-Ketone (PEEK) or Poly-L Lactic Acid (PLLA).

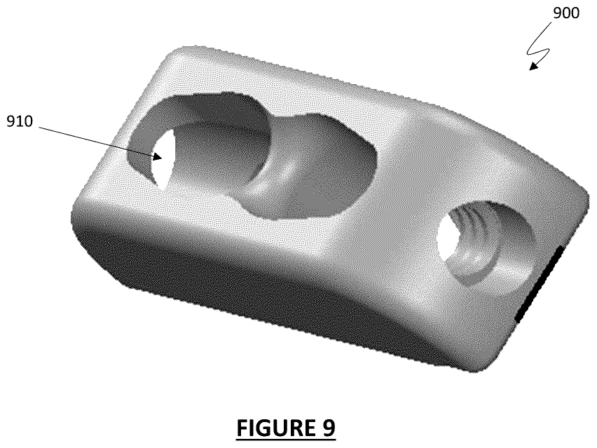

FIG. 9 depicts a perspective view of another alternative embodiment of a button 900 which includes many of the previously described features, but which also includes a distal through hole 910, which hole may be smooth and/or which optionally may include engagement features such as internal threads, hexagonal features, engagement rings or threads, etc. In at least one embodiment, the distal through hole 910 could accommodate a variety of instruments, including to potentially allow the button 900 to be implanted over a guidewire or K-wire (which design may further include a cannulated groove member and/or cannulation through the internal rotatable inserter shaft of the insertion tool, as well as other similar configuration changes, as desired).

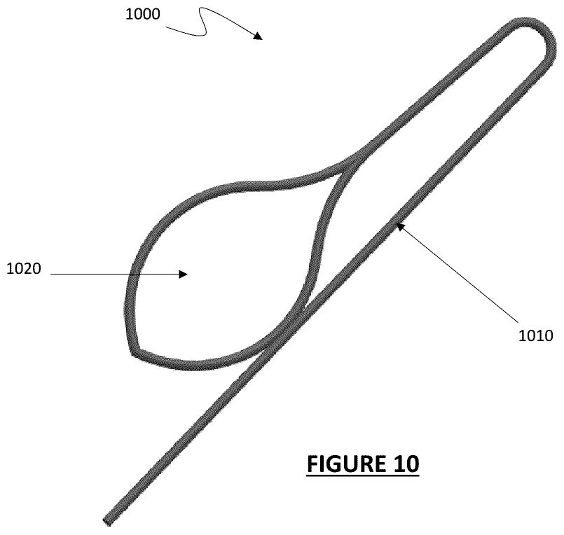

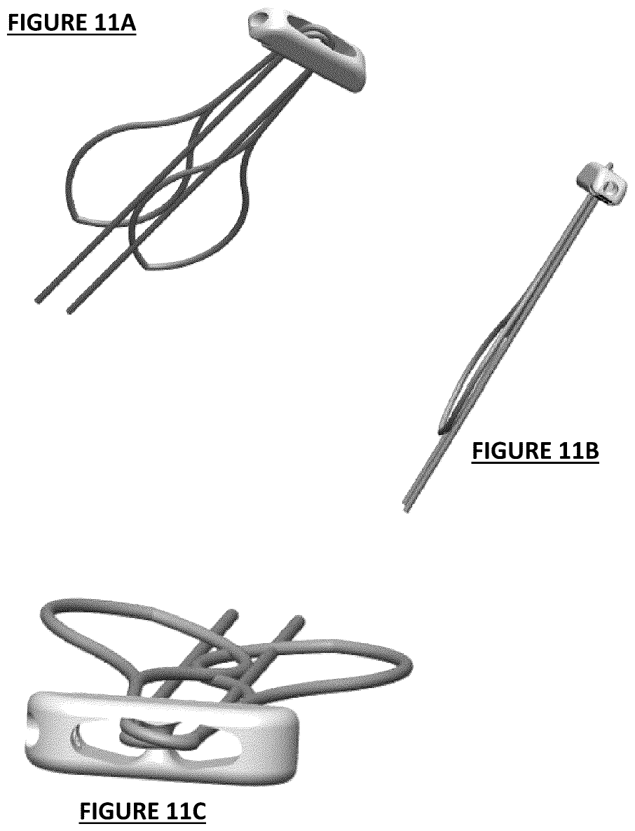

FIG. 10 depicts one exemplary embodiment of a suture loop 1000 or “lasso” (i.e., a Nylon lasso or Nylasso™). In this embodiment, the suture 1010 may comprise a single strand or multi-strand suture or tape wherein a distal end of the suture or suture tape can be looped back upon itself to form an opening or looped section 1020, wherein the suture is adhered or otherwise secured to itself by employing one or more of a variety of weaving or forming techniques, including eye splicing, braiding, adhering or bonding, melt forming, stitching, tying or other connection techniques known in the art. FIGS. 11A through 11C depict various views of a pair of suture loops extending through openings in an exemplary button, which can have a wide range of utility for a variety of surgical procedures.

In various additional embodiments, the disclosed embodiments of buttons and associated system components can optionally include one or more of the following features:
(1) buttons having various shaped proximal and distal ends, including opposing ends having the same, similar or different shapes, including rounded, tapered, flat, angled, pointed, blunt, concave, convex shapes, or various combinations thereof;
(2) buttons having at least two or more suture eyelets, including three, four, five or more longitudinally spaced eyelets, as well as eyelets spaced along a longitudinal axis of the button and/or laterally spaced apart in an adjacent arrangement at a single longitudinal position;
(3) buttons having a threaded and/or unthreaded recess(es) or other engagement features in the proximal and/or distal ends thereof;
(4) buttons that may be preloaded with one or more sutures/suture tapes or that may have no sutures/suture tapes attached;
(5) an inserter shaft having a quick connect or AO-type connection positioned proximally;
(6) an inserter shaft having locating geometry positioned proximally on an outer surface of the shaft;
(7) buttons having various engagement features formed on an outer surface and/or internally, including inner inserter threads positioned on an outer surface of or within the rounded proximal surface of a button;
(8) outer shaft ends having a rounded face and locating geometry that engages with a corresponding rounded face and locating geometry of the button, which desirably allows the button to easily be positioned onto an instrument from a “button caddy” or single/multi-unit packaging;
(9) an inner inserter that extends fully through a handle, AO connection and/or the outer shaft; and/or
(10) a button that is elongated and/or oblong in shape.

At some point in the surgical procedure (or preoperatively, if perioperative imaging has been used), a size, shape and/or length determination could be made to determine an appropriate size and/or shape of the bullet for the given patient anatomy. This selection could include identification of the bony anatomy proximate to the targeted treatment site, as well as surrounding anatomical features, which might influence the physician's choice of bullet size, concavity, curvature and/or angulation. In various embodiments, the characteristics of the fixation device components may be determined in a variety of ways (e.g., X-rays, CT scans, measuring without the guide wire, etc.) preoperatively and/or during the surgical procedure.

As previously noted, implant components could be provided in a kit form in a box or sterilizable holder or containment device (i.e., a “button caddy” having buttons of a variety of shapes, sizes, configurations and/or comprising different materials). The button caddy can allow a user to select a button (i.e., of varying size or shape) of their choice without a need to open a separate box for each desired button size, shape and/or configuration. The button caddy can also allow a user to implement multiple buttons during a single surgical procedure, if necessary.

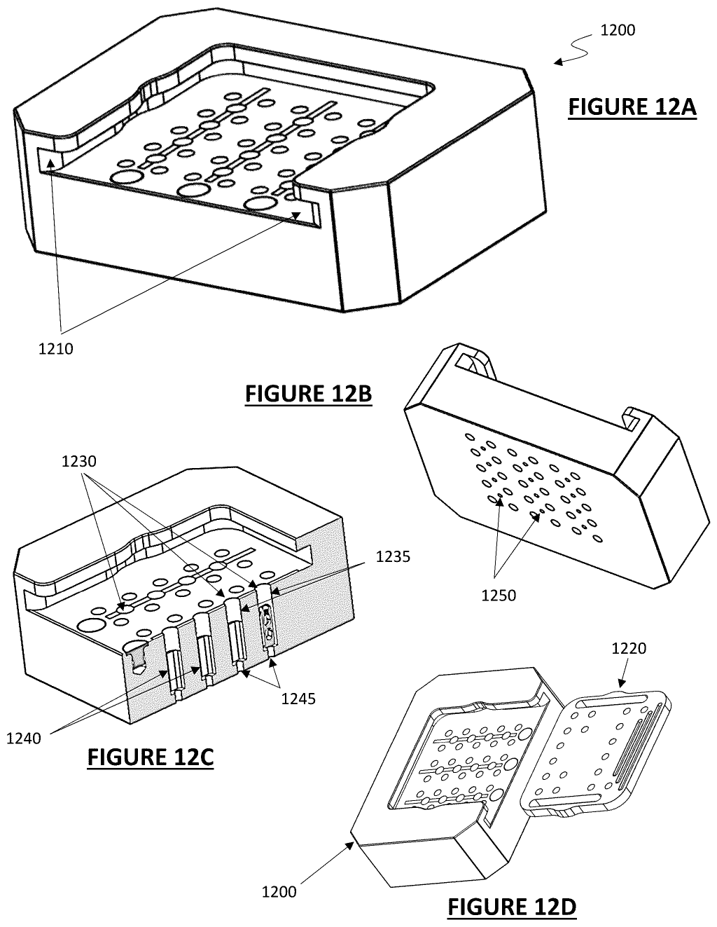

FIGS. 12A through 12C depict one exemplary embodiment of a button caddy 1200. The caddy can include slots 1210 to accommodate a lid or closure 1220 (see FIG. 12D). As best seen in the cross-sectional view of FIG. 12C, the caddy desirably includes a plurality of chambers 1230, with each chamber including a cylindrical upper portion 1235, a rectangular midportion 1240 and a reduced or necked-down lower portion 1245 having a lower opening 1250 in a lower surface of the caddy. In various embodiments the caddy holes can be countersunk and then milled in rectangular shape at lowest ends to properly align buttons. The caddy can be pre-loaded by a manufacturer and provided to a surgeon with a removeable cover provided thereon.

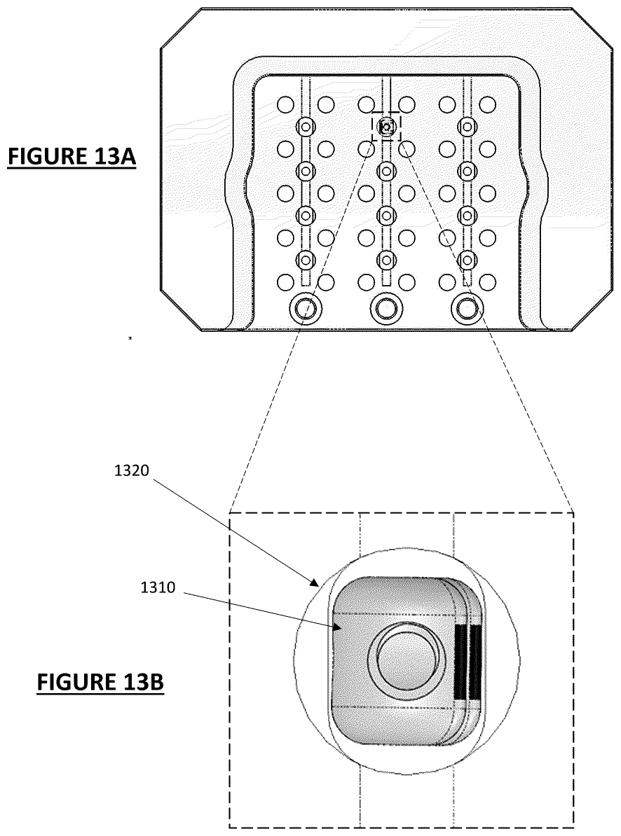

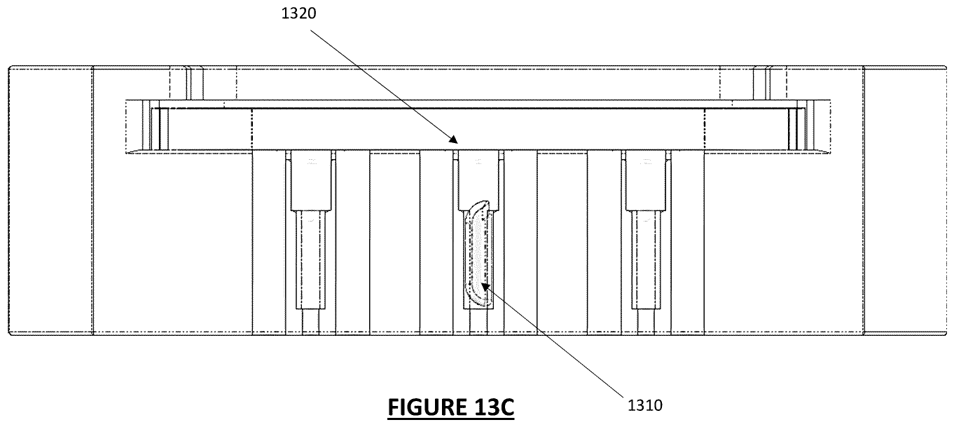

FIGS. 13A and 13B depict top plan views and FIG. 13C depicts a side cross-sectional view of an exemplary implant 1310 positioned within a slot 1320 of one embodiment of a caddy, with FIG. 13C. Desirably, the implant will self-orient within the rectangular midportion section, with the “fishmouth” engagement feature 1330 pointing upwards and extending slightly into the upward into the cylindrical upper portion (e.g., with the counterbore engagement feature facing upwards), which allows the implant to engage with the corresponding inserter and prevents the implant from rotating as the inserter tip is engaged with the counterbore feature of the implant. Such a design desirably allows for the one-way outer inserter to reach and align with the locating feature. In various embodiments, one or more dimensions of the rectangular midportion section can be sized to frictionally retain the implant within the chamber until it is captured or otherwise engaged with the inserted and withdrawn from the caddy using a moderate amount of pulling force.

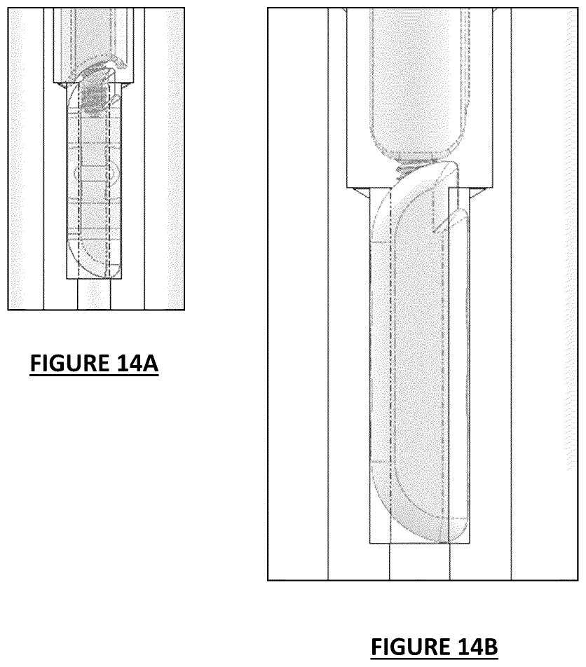

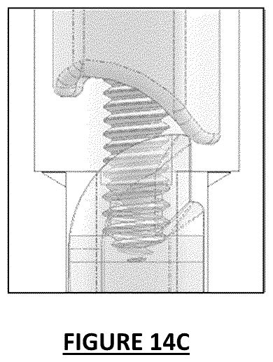

FIG. 14A depicts a cross-sectional partial view of one exemplary embodiment of an inserter and button positioned within a cavity of a caddy. In this embodiment, the fishmouth geometry of the inserter tip is positioned to substantially match the alignment of the button within the cavity, thereby allowing the threaded screw of the inserter to align with and thread into the threaded cavity of the button, connecting the button and inserter for use and allowing the button to be withdrawn out of the cavity (e.g., the pulling force on the inserter can exceed the frictional holding force which retains the button within the caddy). In contrast, FIGS. 14B and 14C depict an example of when the fishmouth geometry of the inserter tip does not substantially match the alignment of the button, wherein the threaded screw of the inserter will desirably not extend far enough to engage with and/or thread into the threaded cavity of the button, thereby preventing and/or inhibiting the button from being removed from the caddy in this alignment. Desirably the fishmouth geometry of the inserter tip will prevent thread engagement so that the button cannot be removed, and no thread engagement occurs, when the inserter and the button are incorrectly orientated.

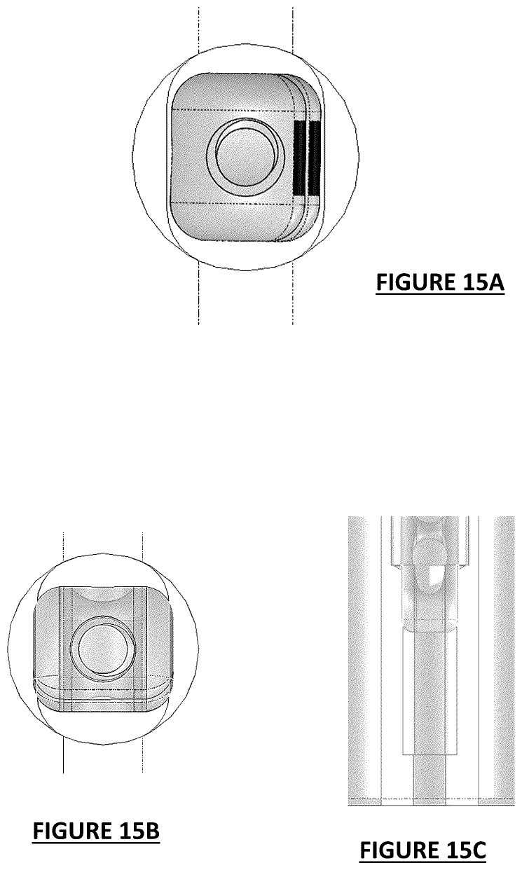

FIG. 15A depicts a top plan view of the exemplary embodiment of a button positioned within a cavity of a caddy, showing a proper insertion orientation (e.g., relative rotational alignment between the button and the cavity). In contrast, FIGS. 15B and 15C depict the button inserted in an incorrect orientation relative to the cavity walls, with FIG. 15B showing interference between the sides of the button and the cavity walls when attempting to load the button incorrectly into the cavity, while FIG. 15C depicts how the button cannot be pushed fully down into the cavity.

In various embodiments, the caddy may be constructed of a softer material such as plastic, while the button may be made of a relatively harder material such as titanium or other metals or ceramics. Desirably, the button(s) will be retained within the caddy by a “press fit” or similar arrangement, which prevents the buttons from falling out of the caddy in even the caddy is inverted while opened or falls to the floor. In various embodiments, the plastic caddy material may even “flex” relative to the metal titanium buttons to hold press fit even better, if desired.

Desirably the caddy and contained buttons can facilitate and/or withstand washing, steam sterilization or other sterilization techniques multiple times, such that a single caddy can be utilized to provide buttons for multiple surgeries and/or reloaded as necessary. The caddy holes can incorporate draining tubes at their bottom surfaces to allow multiple steam sterilization cycles and/or liquid washing, and the drainage holes and other features have been incorporated to facilitate such reuse, sterilization and/or cleaning. In various embodiments the plastic caddy can incorporate multiple through holes that ensure no cracking or uneven heating of the material(s) or contents therein.

The caddy and other components described herein are intended for reuse during multiple surgeries and then recycled by manufacturer. The present designs and features allow one caddy with a plurality of buttons therein to be used serially for multiple surgeries without requiring refilling. Where a caddy is not utilized, a button may be retrieved from sterile packaging and held in one hand while an inserter is placed against the curved face, such that a locating step is met. Then the inner inserter can be threaded on to the button for the procedure, including for selection and/or assembly in a surgical theatre and/or in situ during a surgical procedure. If desired, various components may be removable and replaceable.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein. What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A surgical system for implanting a soft tissue anchoring device into a bone, comprising:
a soft tissue anchor comprising:
an elongated base member having an upper surface and a lower surface with a plurality of longitudinally spaced elongate openings extending through the base member from the upper to the lower surface, the plurality of longitudinally spaced elongate openings space apart by at least one member,
the elongated base member further including a side opening formed at a proximal end of the base member, the side opening having a central axis parallel to a longitudinal axis of the base member, the side opening including an internally threaded section;
the upper surface of the elongated base member further including an external wall portion positioned at the proximal end of the elongated base member which curves downwards towards the lower surface; and
the lower surface including an upwardly notched section at the proximal end of the elongated base member;
an insertion tool having a handle, a cannulated insertion sleeve and a threaded stylet, the cannulated insertion tool having a curved distal tip which engages with the external wall portion positioned at the proximal end of the elongated base member which curves downwards towards the lower surface and a projecting tooth which engages with the upwardly notched section at the proximal end of the elongated base member, a distal tip of the threaded stylet having an externally threaded section which engages with the internally threaded section of the side opening to releasably secure the soft tissue anchor to the insertion tool, and
a containment base for securing a plurality of soft tissue anchors therein.

2. The surgical system of claim 1, wherein when the externally threaded section of the distal tip of the threaded stylet is unthreaded from the internally threaded section of the side opening, the soft tissue anchor can be freely removed from the insertion tool.

3. The surgical system of claim 1, wherein the cannulated insertion sleeve is modularly attached to the handle.

4. The surgical system of claim 1, wherein the threaded stylet can be fully removed from the cannulated insertion sleeve.

5. The surgical system of claim 1, wherein the cannulated insertion sleeve includes at least one externally visible marking indicia that indicates a depth of the soft tissue anchor within the bone.

6. The surgical system of claim 1, wherein the cannulated insertion sleeve includes at least one externally visible marking indicia that indicates when the soft tissue anchor is located fully within a cancellous region of the bone.

7. The surgical system of claim 1, wherein the elongated base member includes at least one flat side wall and the cannulated insertion sleeve includes at least one flat side wall section that is substantially aligned with the at least one flat side wall of the elongated base member.
